(19) United States
(12) Patent Application Publication
(10) Pub. No.: US 2024/0405236 A1
(43) Pub. Date: Dec. 5, 2024

(54) FUEL CELL STACK SENSOR AND A METHOD FOR OPERATING THE SAME

(71) Applicants: HYUNDAI MOTOR COMPANY, Seoul (KR); KIA CORPORATION, Seoul (KR)

(72) Inventor: Gun Ho Kwak, Seoul (KR)

(73) Assignees: HYUNDAI MOTOR COMPANY, Seoul (KR); KIA CORPORATION, Seoul (KR)

(21) Appl. No.: 18/500,498

(22) Filed: Nov. 2, 2023

(30) Foreign Application Priority Data
Jun. 5, 2023 (KR) 10-2023-0072316

(51) Int. Cl. H01M 8/04664; B60L 3/00; B60L 58/30; H01M 8/0432; H01M 8/0444; H01M 8/04492

(52) U.S. Cl. CPC H01M 8/04679; B60L 3/0053; B60L 58/30; H01M 8/04365; H01M 8/04447; H01M 8/04492; H01M 2250/20

(57) ABSTRACT

Disclosed are a fuel cell stack sensor and a method of operating the same. The fuel cell stack sensor has a structure capable of measuring a temperature, a humidity, and a hydrogen concentration inside and outside of a fuel cell stack enclosure including a fuel cell stack by using at least two or more substrates inside the sensor. The fuel cell stack sensory may also determine whether: there is a hydrogen leak and the concentration of the leaked hydrogen; whether there is a leak inside the fuel cell stack enclosure; and whether the fuel cell stack sensor itself is poisoned by volatile organic compounds.

### CROSS-REFERENCE TO RELATED APPLICATION

This application claims the benefit of and priority to Korean Patent Application No. 10-2023-0072316, filed in the Korean Intellectual Property Office on Jun. 5, 2023, the entire contents of which are incorporated herein by reference.

### TECHNICAL FIELD

The present disclosure relates to a fuel cell stack sensor and a method of operating the fuel cell stack sensor.

### BACKGROUND

The statements in this section merely provide background information related to the present disclosure and may not constitute prior art.

As oil reserves continue to dwindle around the world, research into technologies that use green energy as a source of rather than fossil fuels is on the rise, and the automotive sector is no exception. Vehicles that use hydrogen as fuel (hereinafter referred to as ‘hydrogen vehicles’) have been continuously researched recently. In the case of hydrogen vehicles, the vehicles produce water as a by-product and do not emit environmental pollutants. Thus, hydrogen vehicles are expected to become the core of the automobile industry's next-generation.

When air is introduced into the hydrogen vehicle, the introduced air goes through a filtering process to become purified air containing oxygen and can be supplied to the fuel cell stack. Hydrogen stored in advance in the hydrogen storage tank can also be supplied to the fuel cell stack. The supplied oxygen and hydrogen can react to each other in the fuel cell stack to generate water and energy.

Hydrogen gas used as fuel is highly flammable and explosive, and when the concentration is higher than 4%, the possibility of explosion increases significantly, which is fatal to safety. Therefore, in the technology for hydrogen vehicles, it is most important to prevent accidents due to leakage of hydrogen gas, and it is required to pass strict safety standards.

Until now, there are various types of hydrogen sensors such as catalytic combustion, gas thermal conduction, and electrochemical hydrogen sensors. In particular, the gas thermal conduction type hydrogen sensor uses the characteristic that the thermal conductivity of hydrogen gas is significantly higher than that of other gases, and the mixed gas containing hydrogen gas transfers heat faster than the mixed gas without it. Through this difference, it can be determined whether or not hydrogen gas is included in the mixed gas.

### SUMMARY

The present disclosure has been made to solve the above-mentioned problems occurring in the prior art while advantages achieved by the prior art are maintained intact.

An aspect of the present disclosure provides a fuel cell stack sensor usable in an automobile, which not only measures the concentration of hydrogen in a gas mixture around a fuel cell stack but also determines whether the sensor is poisoned by volatile organic compounds (VOCs) and whether or not a leak has occurred inside the fuel cell stack and a method of operating the sensor.

The technical problems to be solved by the present disclosure are not limited to the aforementioned problems. Any other technical problems not mentioned herein should be clearly understood from the following description by those with ordinary skill in the art to which the present disclosure pertains.

According to an aspect of the present disclosure, a fuel cell stack sensor according to an embodiment of the present disclosure is able to determine whether or not there is a leak inside a fuel cell stack and whether the fuel cell stack is operating normally. The fuel cell stack sensor is further able to measure a hydrogen concentration inside the fuel cell stack.

According to an embodiment, a fuel cell stack sensor includes: a first substrate that measures at least one of an external temperature or an external humidity of a fuel cell stack enclosure including a fuel cell stack; a second substrate that measures at least one of the internal temperature or the internal humidity of the fuel cell stack enclosure; and a hydrogen sensing element disposed on the second substrate to detect hydrogen inside the fuel cell stack enclosure.

According to an embodiment, the fuel cell stack sensor may further include: a first temperature element disposed on the first substrate to measure the external temperature of the fuel cell stack enclosure; and a second temperature element disposed on the second substrate to measure the internal temperature of the fuel cell stack enclosure.

According to an embodiment, the fuel cell stack sensor may further include: a first humidity element disposed on the first substrate to measure the external humidity of the fuel cell stack enclosure; and a second humidity element disposed on the second substrate to measure the internal humidity of the fuel cell stack enclosure.

According to an embodiment, the fuel cell stack sensor may further include a measurement controller that receives: data including a first temperature value that is a temperature measured by the first temperature element; data including a second temperature value that is a temperature measured by the second temperature element; data including a first humidity value that is a humidity measured by the first humidity element; and data including a second humidity value that is a humidity measured by the second humidity element.

According to an embodiment, the fuel cell stack sensor may further include a measurement controller that controls: an operation in which the first temperature element measures a first temperature that is the external temperature of the fuel cell stack enclosure; an operation in which the first humidity element measures a first humidity that is the external humidity of the fuel cell stack enclosure; an operation in which the second temperature element measures a second temperature that is the internal temperature of the fuel cell stack enclosure; an operation in which the second humidity element measures a second humidity that is the internal humidity of the fuel cell stack enclosure; and an operation in which the hydrogen sensing element measures a hydrogen concentration inside the fuel cell stack enclosure.

According to an embodiment, the measurement controller may further receive data including a hydrogen concentration value that is a hydrogen concentration detected by the hydrogen sensing element.

According to an embodiment, the measurement controller may calculate a first relative humidity value using data including the first temperature and the first humidity, and calculate a second relative humidity value using data including the second temperature and the second humidity.

According to an embodiment, the measurement controller may determine that volatile organic compound (VOC) poisoning has occurred in the fuel cell stack sensor when the first temperature and the second temperature are the same and the first humidity and the second humidity are different.

According to an embodiment, the measurement controller may compare the first humidity and the second humidity again after a predetermined time has elapsed when the first temperature and the second temperature are the same and the first humidity and the second humidity are the same. Furthermore, the measurement controller may determine that a leak has occurred inside the fuel cell stack enclosure when the first humidity and the second humidity are different after the predetermined time has elapsed.

According to an embodiment, the measurement controller may determine that a leak has occurred inside the fuel cell stack enclosure when the first temperature and the second temperature are different and the first humidity and the second humidity are different.

According to an aspect of the present disclosure, a method of operating a fuel cell stack sensor includes: measuring hydrogen in the fuel cell stack sensor; determining whether the fuel cell stack and the fuel cell stack sensor are operating normally; determining whether a leak has occurred inside the fuel cell stack; and determining whether the fuel cell stack sensor is poisoned by VOC.

According to an embodiment, a method of operating a fuel cell stack sensor includes: an operation of comparing a first temperature value indicating an external temperature of a fuel cell stack enclosure including a fuel cell stack with a second temperature value indicating the internal temperature of the fuel cell stack enclosure; an operation of comparing a first humidity value indicating an external humidity of the fuel cell stack enclosure with a second humidity value indicating the internal humidity of the fuel cell stack enclosure; and determining an operation of the fuel cell stack sensor according to each of result of the operation of comparing the temperatures and the operation of comparing the humidities.

According to an embodiment, when the first temperature value and the second temperature value are the same in the operation of comparing the temperatures, and the first humidity value and the second humidity value are the same in the operation of comparing the humidities, may further include an operation of performing the operation of comparing the humidities again after a predetermined time has elapsed.

According to an embodiment, when the first temperature value and the second temperature value are the same in the operation of comparing the temperatures, and the first humidity value and the second humidity value are different in the operation of comparing the humidities, the determining of the operation of the fuel cell stack sensor may include an operation of determining that the fuel cell stack sensor is poisoned by VOCs.

According to an embodiment, the determining of the operation of the fuel cell stack sensor may include an operation of providing a user with a signal indicating that the fuel cell stack enclosure and the fuel cell stack sensor are normally operating when the first temperature value and the second temperature value are different in the operation of comparing the temperatures and the first humidity value and the second humidity value are the same in the operation of comparing the humidities.

According to an embodiment, the determining of the operation of the fuel cell stack sensor may include an operation of determining that a leak has occurred inside the fuel cell stack enclosure, when the first temperature value and the second temperature value are different in the operation of comparing the temperatures, and the first humidity value and the second humidity value are different in the operation of comparing the humidities.

According to an embodiment, the determining of the operation of the fuel cell stack sensor may include an operation of providing a user with a signal indicating that an inside of the fuel cell stack enclosure and the fuel cell stack sensor are normally operating, when the first temperature value and the second temperature value are the same in the operation of comparing the temperatures, and the first humidity value and the second humidity value are the same and, even after a predetermined time has elapsed, the first humidity value and the second humidity value are the same in the operation of comparing the humidities.

According to an embodiment, the determining of the operation of the fuel cell stack sensor may include operation of determining that a leak has occurred inside the fuel cell stack enclosure, when the first temperature value and the second temperature value are the same in the operation of comparing the temperatures, and the first humidity value and the second humidity value are the same and, after a predetermined time has elapsed, the first humidity value and the second humidity value are different in the operation of comparing the humidities.

According to an embodiment, the method may further include outputting, by the fuel cell stack sensor, a second hydrogen concentration value using the second temperature value, the second humidity value, and a first hydrogen concentration value that is a result of detecting a hydrogen concentration inside the fuel cell stack enclosure, when the determining of the operation of the fuel cell stack sensor includes an operation of providing a user with a signal indicating that an inside of the fuel cell stack enclosure and the fuel cell stack sensor are normally operating.

According to an embodiment, the method may further include generating, by the fuel cell stack sensor, a diagnostic trouble code (DTC) to control a vehicle when the determining of the operation of the fuel cell stack sensor includes an operation of determining that a leak has occurred inside the fuel cell stack enclosure.

According to an embodiment, the method may further include outputting, by the fuel cell stack sensor, a second hydrogen concentration by using the second temperature value, the first humidity value, and a first hydrogen concentration value that is a result of detecting a hydrogen concentration inside the fuel cell stack enclosure when the determining of the operation of the fuel cell stack sensor includes an operation of determining that the fuel cell stack sensor is poisoned by VOC.

### BRIEF DESCRIPTION OF THE DRAWINGS

The above and other objects, features, and advantages of the present disclosure should be more apparent from the following detailed description taken in conjunction with the accompanying drawings:

FIG. 1 is a block diagram showing the internal structure of a hydrogen vehicle including a fuel cell stack sensor according to an embodiment of the present disclosure;

FIG. 2A is a structural diagram showing an embodiment of a fuel cell stack enclosure including the fuel cell stack of FIG. 1 and a structure around a fuel cell stack sensor;

FIG. 2B is a structural diagram showing an embodiment of some components inside the fuel cell stack of FIG. 1;

FIG. 3A is a block diagram showing an embodiment of the internal configuration of the fuel cell stack sensor of FIG. 2A;

FIG. 3B is a structural diagram showing an embodiment of the structure of the fuel cell stack sensor shown in FIG. 3A; and

FIG. 4 is a logic diagram illustrating a method of operating a fuel cell stack sensor according to an embodiment of the present disclosure.

### DETAILED DESCRIPTION

Hereinafter, some embodiments of the present disclosure are described in detail with reference to the drawings. In adding the reference numerals to the components of each drawing, it should be noted that the identical or equivalent component is designated by the identical numeral even when they are displayed on other drawings. Further, in describing embodiments of the present disclosure, a detailed description of well-known features or functions is ruled out in order not to unnecessarily obscure the gist of the present disclosure.

In describing the components of embodiments of the present disclosure, terms such as first and second may be used, and the terms such as first and second are only for distinguishing the component from other components, and unless otherwise specified, the nature, order, or sequence of the corresponding component is not limited by the term. Unless otherwise defined, all terms used herein, including technical or scientific terms, have the same meanings as those generally understood by those with ordinary skill in the art to which the present disclosure pertains. Such terms as those defined in a generally used dictionary are to be interpreted as having meanings equal to the contextual meanings in the relevant field of art and are not to be interpreted as having ideal or excessively formal meanings unless clearly defined as having such in the present application.

When a component, device, element, or the like, of the present disclosure, is described as having a purpose or performing an operation, function, or the like, the component, device, or element should be considered herein as being “configured to” meet that purpose or to perform that operation or function.

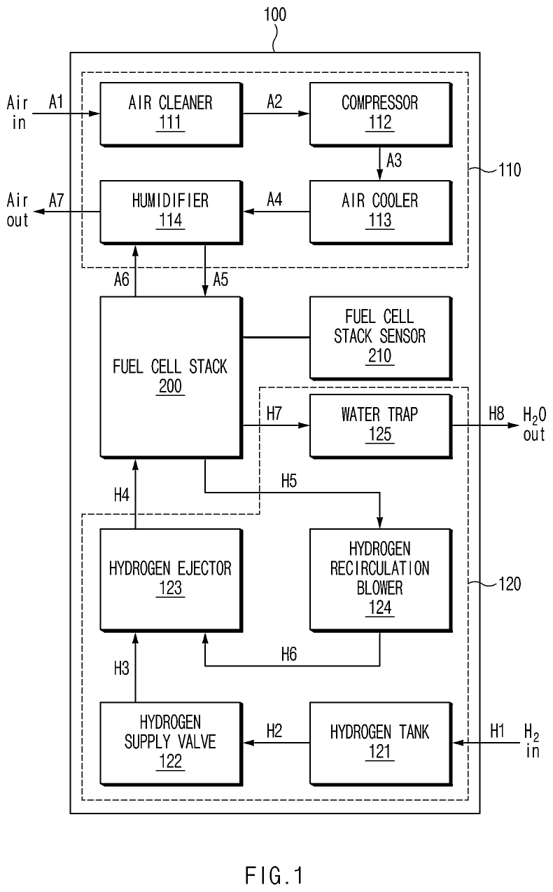

Embodiments of the present disclosure are described below in detail with reference to FIGS. 1-4.

FIG. 1 is a block diagram showing the internal structure of a hydrogen vehicle including a fuel cell stack sensor according to an embodiment of the present disclosure.

Referring to FIG. 1, a hydrogen vehicle 100 may include an air supply system 110, a hydrogen supply system 120, a fuel cell stack 200, and a fuel cell stack sensor 210. The air supply system 110 may include an air cleaner 111, a compressor 112, an air cooler 113, and a humidifier 114, and the hydrogen supply system 120 may include a hydrogen tank 121, a hydrogen supply valve 122, a hydrogen ejector 123, a hydrogen recirculation blower 124, and a water trap 125.

The air cleaner 111 may include a filter structure capable of filtering out foreign substances when air A1 containing oxygen (O₂) is introduced from the outside of the hydrogen vehicle 100. The foreign substances may include ultrafine dust having a size of 2.5 μm or less, and the filter structure may remove 99% or more of the ultrafine dust. The percentage figure (99%) is just an example. The scope of the present disclosure is not limited to the percentage figure. When the filter structure filters out the foreign substances, the yield of electrical energy generation reaction in the fuel cell stack 200 may be improved. The air cleaner 111 may send purified air A2 containing O₂ to the compressor 112. The electrical energy generation reaction may be a reaction in which oxygen (O₂) and hydrogen (H₂) react to generate electrical energy and water (H₂O) (hereinafter referred to as an electrical energy generation reaction).

The compressor 112 may serve to receive the purified air A2 from the air cleaner 111, compress the purified air A2 with high efficiency, and supply the purified air A2 to the air cooler 113. The compressor 112 may output compressed air by rotating the purified air A2 introduced into the compressor 112 at high speed with a high-speed motor or the like. The compressor 112 may increase the pressure of the purified air A2 to 3.0 bar. As the pressure of the supplied air increases, the supply amount of O₂ contained in the air may further increase. The pressure value (3.0 bar) is an example and the scope of the present disclosure is not limited to the pressure value.

The high-pressure air A3 received by the air cooler 113 from the compressor 112 may have high-temperature and dry characteristics. The air cooler 113 may cool the high-pressure air A3 which has risen to about a temperature of 100° C. to 150° C. in the compression process. More specifically, a normal operating temperature range of the fuel cell stack 200 may be 60° C. to 80° C., and the air cooler 113 may cool the temperature of the high-pressure air A3 within the normal operating temperature range. The air cooler 113 may supply cooled air A4 to the humidifier 114 after the cooling is finished.

The humidifier 114 may receive the cooled air A4, which has been cooled to an appropriate temperature, from the air cooler 113, and may adjust the humidity of the cooled air A4 before supplying the cooled air A4 into the fuel cell stack 200. The adjusted humidity may be, for example, relative humidity (RH) of 70% RH. To stably generate electrical energy inside the fuel cell stack 200, it is desired to supply air containing moisture to the fuel cell stack 200 and to supply air containing moisture, the humidifier 114 may supply humidified air A5 to the fuel cell stack 200. The humidifier 114 may additionally remove a small amount of ultra-fine dust that is not removed by the air cleaner 111 and remains in the process of humidifying the cooled air A4.

In the fuel cell stack 200, oxygen (O₂) included in the humidified air A5 supplied from the humidifier 114 may be used as a reactant for the electrical energy generation reaction that generates electrical energy. According to the principle of chemical equilibrium, unused oxygen (O₂) may exist, and air (hereinafter, unused air A6) containing the unused oxygen (O₂) may move from the fuel cell stack 200 back to the humidifier 114. In addition, the humidifier 114 may discharge oxygen (O₂) contained in the unused air A6 to the outside of the hydrogen vehicle 100.

The hydrogen tank 121 may receive and store a large amount of hydrogen H1 from a charging station capable of charging hydrogen (H₂). Hydrogen stored in the hydrogen tank 121 may pass through the hydrogen supply valve 122 while being supplied to the fuel cell stack 200.

The amount of hydrogen (H₂) supplied to the hydrogen supply valve 122 may be regulated by the hydrogen supply valve 122 when being supplied to the hydrogen ejector 123. The hydrogen ejector 123 may supply hydrogen including hydrogen H3 passed through the hydrogen supply valve 122 and hydrogen H6 supplied from the hydrogen recirculation blower 124 to the fuel cell stack 200.

The hydrogen H4 supplied to the fuel cell stack 200 may be subject to the electrical energy generation reaction with the oxygen (O₂) contained in the air A5 supplied from the humidifier 114 in the fuel cell stack 200 to produce water (H₂O). Among the hydrogen H4 supplied to the fuel cell stack 200, not all hydrogen (H₂) may not participate in the electrical energy generation reaction, and some unreacted hydrogen H5 may exist according to the principle of equilibrium. The unreacted hydrogen H5 may move to the hydrogen recirculation blower 124.

The hydrogen recirculation blower 124 may serve as an intermediate base for resupplying the unreacted hydrogen H5 to the fuel cell stack 200 again when the unreacted hydrogen H5 is introduced from the fuel cell stack 200. The hydrogen recirculation blower 124 may supply the unreacted hydrogen H5 to the hydrogen ejector 123. In another embodiment of the present disclosure, when the hydrogen supply system 120 does not use the hydrogen recirculation blower 124, the unreacted hydrogen H5 may move to the hydrogen ejector 123. In this case, the hydrogen ejector 123 may supply the hydrogen H3 that has passed through the hydrogen supply valve 122 and the unreacted hydrogen H5 back to the fuel cell stack 200.

Water H7, which is a by-product generated through the electrical energy generation reaction in the fuel cell stack 200, may move to the water trap 125. The water trap 125 may discharge the introduced water H7 to the outside of the hydrogen vehicle 100. In the process of discharging, the water H7 may be discharged through the humidifier 114.

The fuel cell stack sensor 210 may be connected to the fuel cell stack 200, and may be connected to the inside of the fuel cell stack 200 through a predetermined passage connected to a route through which air is introduced into the fuel cell stack 200. The fuel cell stack sensor 210 may measure the temperature and/or humidity of the humidified air A5 introduced into the fuel cell stack 200. The fuel cell stack sensor 210 may also measure the concentration of hydrogen. More specifically, unreacted hydrogen H5 among the hydrogen H4 supplied to the fuel cell stack 200 by the hydrogen supply system 120 may leak into the fuel cell stack 200 for some reason without moving to the hydrogen recirculation blower 124, and the fuel cell stack sensor 210 may measure the concentration of the leaked hydrogen. In addition, the fuel cell stack sensor 210 may also measure the external temperature and humidity of the fuel cell stack 200, and the internal humidity and external humidity of the fuel cell stack 200 measured by the fuel cell stack sensor 210 may be absolute humidity.

The description given with reference to FIG. 1 is only an example. Other components not mentioned in the description may additionally exist in the air supply system 110 or the hydrogen supply system 120.

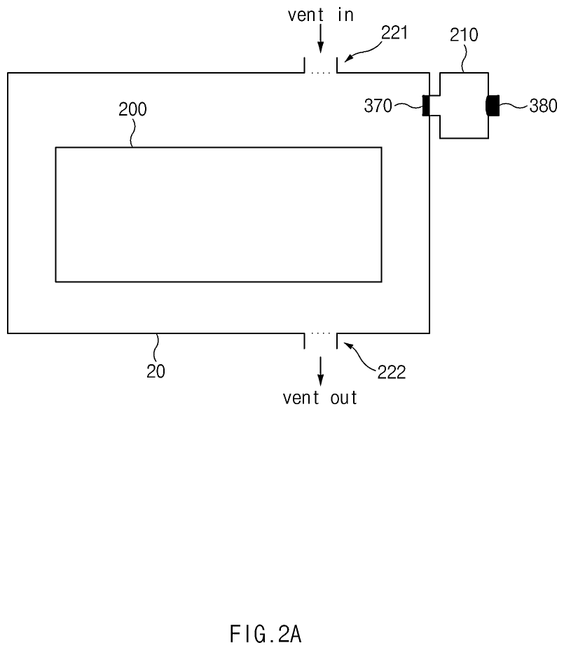

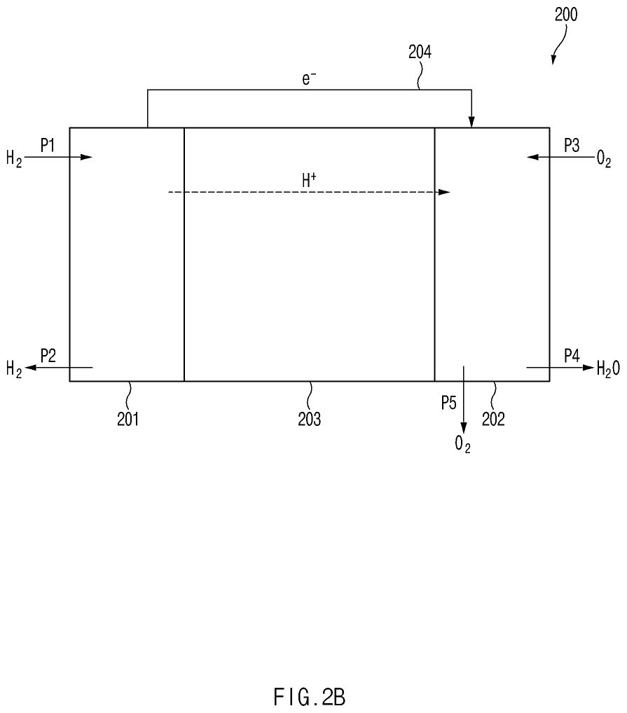

FIG. 2A is a structural diagram showing an embodiment of a fuel cell stack enclosure including the fuel cell stack of FIG. 1 and a structure around a fuel cell stack sensor.

FIG. 2B is a structural diagram showing a part of the internal configuration of the fuel cell stack of FIG. 1.

Referring to FIGS. 1 and 2A, the fuel cell stack 200 of FIG. 1 and the fuel cell stack 200 of FIG. 2 may be substantially the same. A fuel cell stack enclosure 20 may include the fuel cell stack 200, and may include an inlet portion 221 of a fuel cell stack enclosure flow route through which air is vented into the fuel cell stack enclosure 20, and an outlet portion 222 of the fuel cell stack enclosure flow route through which air introduced into the fuel cell stack enclosure 20 is vented out. The inlet 221 of the flow route through which air is introduced into the fuel cell stack enclosure 20 may be different from a portion through which the humidified air A5 that has passed through the humidifier 114 is introduced into the fuel cell stack 200 in a path through which external air is introduced into the fuel cell stack 200 by the air supply system 110.

Referring to FIGS. 1 and 2B, the fuel cell stack 200 of

FIG. 2B may include a region 201 including an cathode (hereinafter referred to as an cathode region), a region 202 including a anode (hereinafter referred to as a anode region), and a region 203 including an electrolyte (hereinafter referred to as an electrolyte region). The cathode region 201 may correspond to an area to which the hydrogen H4 is supplied when the hydrogen H4 that had passed through the hydrogen ejector 123 is supplied to the fuel cell stack 200 by the hydrogen supply system 120. The anode region 202 may correspond to an area to which the air A5 is supplied when the air A5 that has passed through the humidifier 114 is supplied to the fuel cell stack 200 by the air supply system 110. The electrolyte region 203 may be a space in which H+ is able to move toward the anode region 202 when hydrogen participates in the electrical energy generation reaction in the fuel cell stack 200, other than unused hydrogen P2 among the hydrogen P1 supplied to the cathode region 201, is decomposed into H+ and e−. The cathode region 201 and the anode region 202 may be physically separated from each other, and the electrolyte region 203 may exist between the cathode region 201 and the anode region 202.

Referring to FIGS. 2A and 2B, the fuel cell stack sensor 210 may be adjacent to the fuel cell stack enclosure 20, and an inner flow hole 370 may be positioned between the fuel cell stack enclosure 20 and the fuel cell stack sensor 210. The inner flow hole 370 may be positioned in a region closer to the inlet 221 of the flow route than to the outlet 222 of the flow route of the fuel cell stack enclosure 20. The inner flow hole 370 may serve as a passage through which air introduced into the fuel cell stack enclosure 20 through the inlet 221 of the flow route flows into the fuel cell stack sensor 210. The fuel cell stack sensor 210 may measure the temperature and/or humidity of air introduced into the fuel cell stack enclosure 20 (hereinafter, “temperature and/or humidity of air inside the fuel cell stack enclosure 20”). The fuel cell stack sensor 210 may measure the concentration of leaked hydrogen if there is leaked hydrogen in the fuel cell stack enclosure 20.

The hydrogen Pl supplied to the cathode region 201 may be hydrogen H4 supplied to the fuel cell stack 200 of the hydrogen supply system 120 (see FIG. 1). At least a part of the hydrogen (H₂) introduced into the cathode region 201 may be decomposed into two protons (H+) and two electrons (e−). The protons (H+) may move to the electrolyte region 203 and then move from the electrolyte region 203 to the anode region 202. The electrons (e−) may move from the cathode region 201 to the anode region 202 through a predetermined electric wire 204.

A part of the hydrogen (H₂) may be discharged to the outside of the cathode region 201 without being decomposed in the cathode region 201, and the undecomposed hydrogen P2 (hereinafter referred to as unused hydrogen P2) may include hydrogen H5 that moves to the hydrogen recirculation blower 124 of the hydrogen supply system 120 (FIG. 1).

Oxygen P3 supplied to the anode region 202 may be included in air A5 supplied from the humidifier 114 of the air supply system 110 (FIG. 1) to the fuel cell stack 200. In the anode region 202, oxygen (O₂), the protons (H+) moved from the electrolyte region 203 and the electrons (e−) moved through the predetermined wire 204 may react (hereinafter, electrical energy generation reaction) to form water (H₂O).

There may be unused oxygen P5 not used in the reaction to form the water H₂O, and the unused oxygen P5 may be discharged to the outside of the anode region 202. The unused oxygen P5 discharged to the outside of the anode region 202 may be included in air A6 that moves from the fuel cell stack 200 of the air supply system 110 (FIG. 1) to the humidifier 114 again. The water P4 formed by the electrical energy generation reaction may be discharged to the outside of the anode region 202, and the water P4 discharged to the outside of the anode region 202 may also be discharged to the outside through the humidifier 114 of the air supply system 110 (FIG. 1).

In the anode region 202, when there is a special circumstance such as when airtight is broken in the internal structure of the fuel cell stack 200, hydrogen (H₂) may exist inside the fuel cell stack 200 and the fuel cell stack enclosure 20, and the fact that the fuel cell stack sensor 210 connected to the fuel cell stack enclosure 20 detects hydrogen H₂ may mean that a leak of hydrogen H₂ has occurred. The fuel cell stack sensor 210 may measure the concentration of the leaked hydrogen H₂.

As described above, the fuel cell stack sensor 210 may measure the temperature and/or humidity of air inside the fuel cell stack enclosure 20 introduced through the inner flow hole 370 located between the fuel cell stack enclosure 20 and the fuel cell stack sensor 210 and also measure the concentration of hydrogen. The fuel cell stack sensor 210 may further measure the temperature and/or humidity of the air outside the fuel cell stack enclosure 20 introduced through an outer flow hole 380.

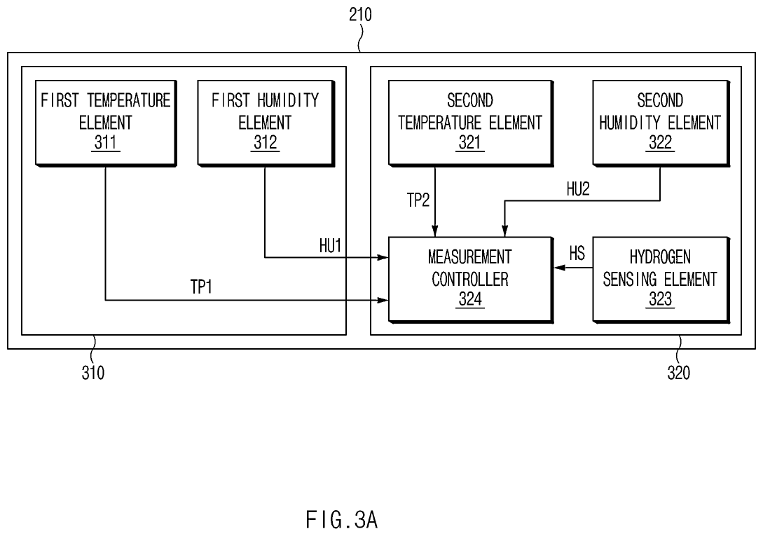

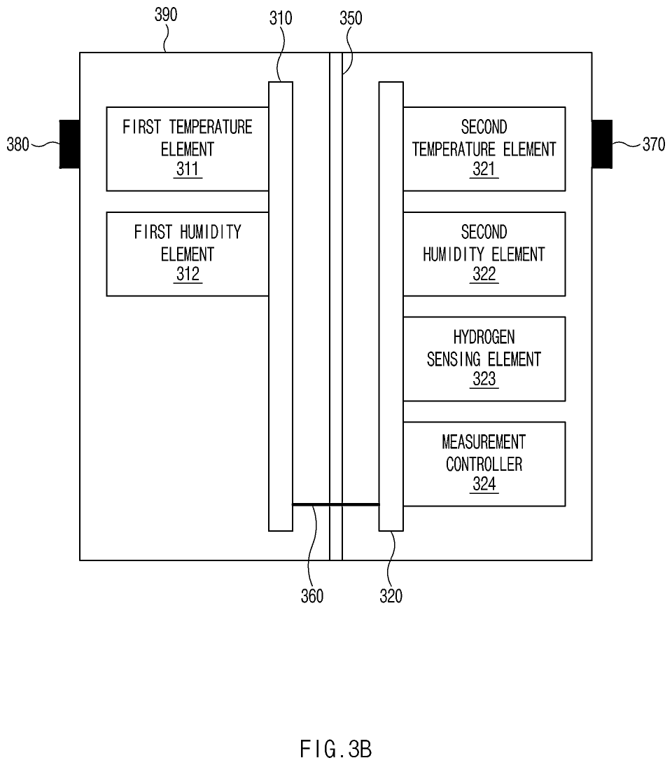

FIG. 3A is a block diagram showing an embodiment of the internal configuration of the fuel cell stack sensor of FIG. 2A. FIG. 3B is a structural diagram showing an embodiment of a structure of the fuel cell stack sensor shown in FIG. 3A.

Referring to FIGS. 2A, 3A, and 3B, the fuel cell stack sensor 210 may include: a first substrate 310 on which elements capable of measuring the temperature and/or humidity of air outside the fuel cell stack enclosure 20 are disposed; a second substrate 320 on which elements capable of measuring the temperature and/or humidity of the air inside the fuel cell stack enclosure 20 are disposed; a separation plate 350 that physically separates the first substrate 310 and the second substrate 320; an electric wire 360 that electrically connects the first substrate 310 and the second substrate 320; the inner flow hole 370 that is a passage through which air inside the fuel cell stack enclosure 20 is able to flow in; the outer flow hole 380 through which air outside the fuel cell stack enclosure 20 is able to flow in; and a fuel cell stack sensor case 390. The inner flow hole 370 of FIG. 3B may be substantially the same as the inner flow hole 370 of FIG. 2A. The inner flow hole 370 may be an air passage located between the fuel cell stack enclosure 20 and the fuel cell stack sensor 210. The outer flow hole 380 of FIG. 3B may be substantially the same as the outer flow hole 380 of FIG. 2A.

A first temperature element 311 and a first humidity element 312 may be disposed on the first substrate 310. A second temperature element 321, a second humidity element 322, a hydrogen sensing element 323, and a measurement controller 324 may be disposed on the second substrate 320.

Referring to FIGS. 2A and 3B, the first substrate 310 and the second substrate 320 may be disposed in parallel to face each other. The first substrate 310 and the second substrate 320 may be positioned parallel to a surface which the fuel cell stack enclosure 20 and the fuel cell stack sensor 210 face, and the first substrate 310 may be positioned further away from the fuel cell stack enclosure 20 than the second substrate 320. (Hereinafter, ‘positional relationship’ between the first substrate 310 and the second substrate 320) The present disclosure is not limited to the configuration in which the first substrate 310 and the second substrate are positioned in parallel to face each other. When assuming that the two spaces separated by the separation plate 350 in the fuel cell stack sensor 210 are the first space and the second space, the first substrate 310 may be positioned freely in the first space and the second substrate 320 may be positioned freely in the second space. The above parallel positional relationship may correspond to an embodiment. The first space may be adjacent to the outer through hole 380, and the second space may be adjacent to the inner through hole 370. According to the positional relationship between the first substrate 310 and the second substrate 320, the first temperature element 311 disposed on the first substrate 310 may measure an external temperature of the fuel cell stack enclosure 20. According to the positional relationship between the first substrate 310 and the second substrate 320, the first humidity element 312 disposed on the first substrate 310 may measure an external humidity of the fuel cell stack enclosure 20. The humidity measured by the first humidity element 312 may be absolute humidity.

According to the positional relationship between the first substrate 310 and the second substrate 320, the second temperature element 321 disposed on the second substrate 320 may measure the internal temperature of the fuel cell stack enclosure 20. According to the positional relationship between the first substrate 310 and the second substrate 320, the second humidity element 322 disposed on the second substrate 320 may measure the internal humidity of the fuel cell stack enclosure 20. According to the positional relationship between the first substrate 310 and the second substrate 320, the hydrogen sensing element 323 disposed on the second substrate 320 may measure the concentration of hydrogen inside the fuel cell stack 200. The humidity measured by the second humidity element 322 may be absolute humidity.

Referring to FIGS. 3A and 3B, the measurement controller 324 may be disposed on the second substrate 320. In the present disclosure, the measurement controller 324 is not limited to being included in the second substrate 320. For example, another third substrate (not shown) different from the first substrate 310 and the second substrate 320 may be provided, and the measurement controller 324 may be located on the third substrate.

Because the temperature and/or humidity of the external air of the fuel cell stack enclosure 20 is measured by the first substrate 310, the temperature and/or humidity of the internal air of the fuel cell stack enclosure 20 is measured by the second substrate 320, the separation plate 350 may block the movement of materials and heat between the first substrate 310 and the second substrate 320.

The electrical wire 360 may be configured to transmit data including the temperature and/or humidity of the external air of the fuel cell stack enclosure 20 measured by the first substrate 310 to the measurement controller 324 that may be disposed on the second substrate 320. When the measurement controller 324 is disposed on the first substrate 310, data including the temperature and/or humidity of the internal air of the fuel cell stack enclosure 20 measured by the second substrate 320 may be transmitted to the measurement controller 324 through the electrical wire 360. The electrical wire 360 may pass through the separation plate 350.

The inner flow hole 370 may serve as a passage through which internal air of the fuel cell stack enclosure 20 is introduced into the fuel cell stack sensor 210, and the outer flow hole 380 may serve as a passage through which external air of the fuel cell stack enclosure 20 is introduced into the fuel cell stack sensor 210. The interior of the fuel cell stack sensor case 390 may be divided by the separation plate 350, and all areas of the surface except for the inner flow hole 370 and the outer flow hole 380 may block the movement of materials. At least one inner flow hole 370 and at least one outer flow hole 380 may exist, and at least one inner flow hole 370 may be disposed at a position where an air movement path is possible between the fuel cell stack enclosure 20 and the fuel cell stack sensor 210, and at least one outer flow hole 380 may be disposed on the surface of the fuel cell stack sensor case 390 adjacent to the first space rather than the second space adjacent to the inner flow hole 370.

Hereinafter, an embodiment of the present disclosure in which the measurement controller 324 is included in the second substrate 320 is described with reference to FIGS. 3A and 3B.

The temperature of the external air of the fuel cell stack enclosure 20 measured by the first temperature element 311 is referred to as a first temperature, and the value thereof is referred to as a first temperature value. In one embodiment of the present disclosure, the first temperature element 311 disposed on the first substrate 310 may transmit data TP1 including the first temperature value (hereinafter referred to as first temperature data) to the measurement controller 324 which may be disposed on the second substrate 320 through the electrical wire 360 connecting the first substrate 310 and the second substrate 320.

The first temperature may be the external temperature of the fuel cell stack enclosure. The second temperature may be the internal temperature of the fuel cell stack enclosure. The first humidity may be the external humidity of the fuel cell stack enclosure. The second humidity may be the internal humidity of the fuel cell stack enclosure.

The humidity of the external air of the fuel cell stack enclosure 20 measured by the first humidity element 312 is referred to as a first humidity, and the value thereof is referred to as a first humidity value. In one embodiment of the present disclosure, the first humidity element 312 disposed on the first substrate 310 may transmit data HU1 including the first humidity value (hereinafter referred to as first humidity data) to the measurement controller 324, which may be disposed on the second substrate 320, through the electric wire 360 connecting the first substrate 310 and the second substrate 320.

The temperature of the internal air of the fuel cell stack enclosure 20 measured by the second temperature element 321 is referred to as a second temperature, and the value thereof is a second temperature value. In one embodiment of the present disclosure, the second temperature element 321 disposed on the second substrate 320 may transmit data TP2 including the second temperature value (hereinafter referred to as second temperature data) to the measurement controller 324 which may be disposed on the second substrate 320.

The humidity of the internal air of the fuel cell stack enclosure 20 measured by the second humidity element 322 is referred to as a second humidity, and the value thereof is a second humidity value. In one embodiment of the present disclosure, the second humidity element 322 disposed on the second substrate 320 may transmit data HU2 including the second temperature value (hereinafter referred to as second humidity data) to the measurement controller 324 which may be disposed on the second substrate 320.

A hydrogen concentration value of the internal air of the fuel cell stack enclosure 20 measured by the hydrogen sensing element 323 is referred to as a first hydrogen concentration value. In one embodiment of the present disclosure, the hydrogen sensing element 323 of the second substrate 320 may transmit data HS including the first hydrogen concentration value (hereinafter referred to as hydrogen concentration data) to the measurement controller 324 which may be disposed on the second substrate 320.

The first humidity element 312 disposed on the first substrate 310 and the second humidity element 322 disposed on the second substrate 320 may be humidity elements that measure humidity using a moisture-sensitive material. The moisture-sensitive material may accommodate water molecules existing in the air, and physical properties of the moisture-sensitive material may change when the water molecules are adsorbed onto the moisture-sensitive material. As an example of a change in physical properties of the moisture-sensitive material, electric capacity may increase when water molecules are adsorbed onto the moisture-sensitive material. When molecules corresponding to volatile organic compounds (VOCs) are present in the air, the moisture-sensitive material may also accommodate the VOC molecules. When the VOC molecules are adsorbed onto the moisture-sensitive material, physical properties of the moisture-sensitive material may change, similar to the case where water molecules are adsorbed onto the moisture-sensitive material. For example, the moisture-sensitive material may have an increased electric capacity when VOCs are adsorbed onto the moisture-sensitive material. The humidity element for measuring humidity using the moisture-sensitive material may measure absolute humidity.

Absolute humidity is the humidity that expresses the mass of water vapor contained in a unit volume of air, and relative humidity is the humidity calculated by dividing the actual vapor pressure in the air by the saturated vapor pressure. The saturated vapor pressure may change according to the temperature, and the range of change of the saturated vapor pressure may increase as the temperature increases.

The hydrogen sensing element 323 may be a hydrogen sensing element utilizing the fact that the thermal conductivity of hydrogen is higher than that of other molecules. For example, the hydrogen sensing element 323 may include a heater (not shown), a third temperature element (not shown), and a fourth temperature element (not shown), and the heater temperature element to a constant may heat the third temperature. Heat generated from the heater may be transferred to the fourth temperature element by a mixed gas existing between the heater and the fourth temperature element. Due to the heat transfer and heating mechanism, the third temperature element may provide a reference temperature and the fourth temperature element may provide a variable temperature. The hydrogen sensing element 323 may detect whether hydrogen is included in the mixed gas by comparing the temperature of the third temperature element and the temperature of the fourth temperature element. More specifically, the hydrogen sensing element 323 may measure the temperature of the fourth temperature element higher when hydrogen is introduced into the surrounding mixed gas than when hydrogen is not introduced, and the hydrogen sensing element 323 may measure the concentration of hydrogen in the mixed gas in consideration of the predetermined thermal conductivity of hydrogen and the change in temperature of the second temperature element. The predetermined thermal conductivity of hydrogen may be, for example, the thermal conductivity of hydrogen at room temperature (about 25° C.) and normal humidity (50% RH). It may be assumed that the hydrogen concentration measured by the hydrogen sensing element 323 is a first hydrogen concentration value.

In the heat transfer, the thermal conductivity of the hydrogen may change due to the temperature and humidity of the mixed gas containing the introduced hydrogen gas. Therefore, the thermal conductivity of hydrogen in the actual gas and the predetermined thermal conductivity of hydrogen may be different. The first hydrogen concentration value measured by the hydrogen sensing element 323 may need to be corrected according to the actual temperature and actual humidity. A parameter reflecting the actual temperature and actual humidity may be a relative humidity value.

The measurement controller 324 may calculate a first relative humidity value using the first temperature value and the first humidity value, and calculate a second relative humidity value using the second temperature value and the second humidity value. In performing the necessary correction, the measurement controller 324 may perform a process of correcting the first hydrogen concentration value using the second temperature value and the calculated first relative humidity value or second relative humidity value. The measurement controller 324 may calculate a second hydrogen concentration value by correcting the first hydrogen concentration value.

Referring to FIGS. 2A and 3A, the measurement controller 324 may determine whether the first temperature value and the second temperature value are the same by comparing the first temperature value and the second temperature value. The measurement controller 324 may determine whether the first humidity value and the second humidity value are the same by comparing the first humidity value and the second humidity value. The measurement controller 324 may determine whether the fuel cell stack 200 and/or the fuel cell stack sensor 210 is normally operating by using a result of the comparison of the two temperature values and a result of the comparison of the two humidity values.

When the first temperature value is equal to the second temperature value, the measurement controller 324 may determine whether the first humidity value and the second humidity value are the same by comparing the first humidity value and the second humidity value. When the first humidity value is also equal to the second humidity value, the measurement controller 324 may redetermine whether the first humidity value and the second humidity value are the same by comparing the first humidity value and the second humidity value after a predetermined time has elapsed. As a result of the redetermination, when the first humidity value is equal to the second humidity value, the measurement controller 324 may provide a signal indicating that the fuel cell stack 200 and/or the fuel cell stack sensor 210 is operating normally to a user.

When the first temperature value and the second temperature value are different, the measurement controller 324 may determine whether the hydrogen vehicle 100 (FIG. 1) is restarted immediately after driving. The measurement controller 324 may determine whether the first humidity value and the second humidity value are the same by comparing the first humidity value and the second humidity value. When the first humidity value is equal to the second humidity value measurement controller 324 may provide a signal indicating that the fuel cell stack 200 and/or the fuel cell stack sensor 210 is operating normally to the user.

The measurement controller 324 may determine that the inside of the fuel cell stack sensor 210 is poisoned by VOCs when the first temperature value is equal to the second temperature value and the first humidity value and the second humidity value are different, and may provide the fact that the fuel cell stack sensor 210 is poisoned by VOCs to the user.

When the first temperature value is equal to the second temperature value and the first humidity value is equal to the second humidity value, the measurement controller 324 may redetermine whether the first humidity value and the second humidity value are the same by comparing the first humidity value and the second humidity value after a predetermined time has elapsed. As a result of the redetermination, when the first humidity value and the second humidity value are different, the measurement controller 324 may determine that a leak has occurred inside the fuel cell stack 200 and/or the fuel cell stack enclosure 20, and provide the fact of the internal leak to the user.

When the first temperature value and the second temperature value are different and the first humidity value and the second humidity value are different, the measurement controller 324 may determine that a leak has occurred inside the fuel cell stack 200 and/or the fuel cell stack enclosure 20 and may provide the fact of the internal leak to the user.

In determining whether the values are the same, the meaning of ‘the same’ may be a concept also including a case where a difference (error) between the first temperature value (or first humidity value) and the second temperature value (or second humidity value) is within a predetermined error range.

The measurement controller 324 may transmit signals for controlling the operations of the first temperature element 311, the first humidity element 312, the second temperature element 321, the second humidity element 322, and the hydrogen sensing element 323, respectively.

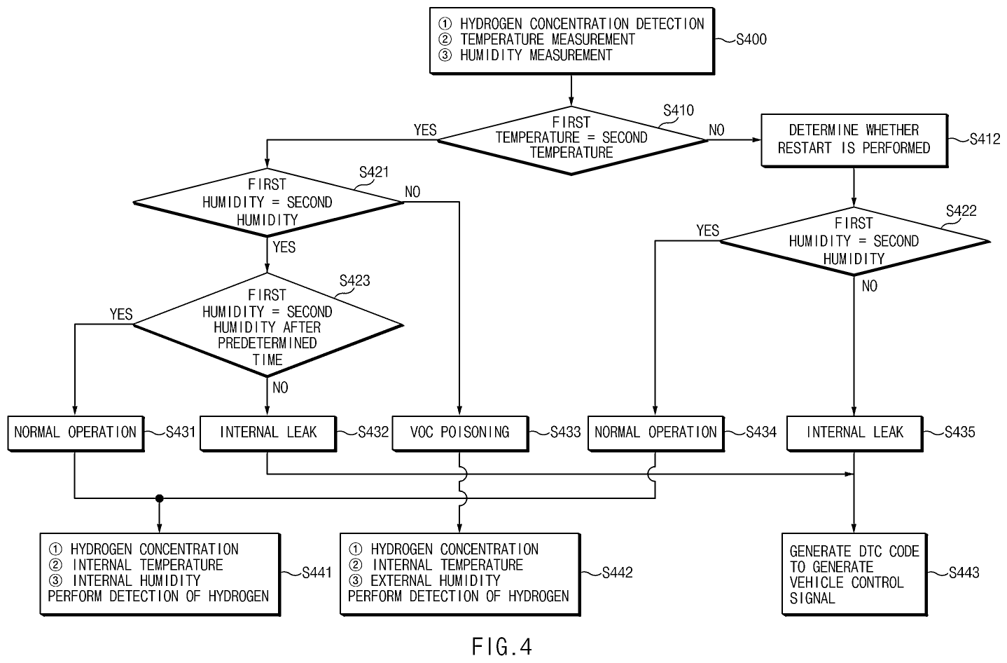

FIG. 4 is a logic diagram illustrating a method of operating a fuel cell stack sensor according to an embodiment of the present disclosure.

FIG. 4 shows a method of operating the fuel cell stack sensor 210 of FIGS. 1, 2A, 2B, 3A, and 3B, and the internal configuration for controlling the operation may be, for example, the measurement controller 324. In the present disclosure, the control of the operation of the fuel cell stack sensor 210 cannot be construed as limited to being performed only by the measurement controller 324, and for example, the control of the operation may be performed by two or more internal components of the fuel cell stack sensor 210.

Details to be described below are specific descriptions of an embodiment of the method of operating the fuel cell stack sensor 210 and are related to an embodiment in which the measurement controller 324 controls the operation.

Referring to FIG. 1, when the hydrogen vehicle 100 of FIG. 1 is started, the power of the fuel cell stack sensor 210 may be turned on. When the engine of the hydrogen vehicle 100 (FIG. 1) is turned off, the power of the fuel cell stack sensor 210 may be turned off. The fuel cell stack sensor 210 may continuously operate while the power is turned on.

Referring to FIGS. 2A, 3A, and 4, the method of operating the fuel cell stack sensor 210 may include performing hydrogen concentration measurement, temperature measurement, and humidity measurement (S400, hereinafter referred to as ‘operation of performing measurement’). In the operation of performing measurement (S400), the temperature measurement may include measuring the temperature of the internal air of the fuel cell stack enclosure 20 and measuring the temperature of the external air of the fuel cell stack enclosure 20. In the operation of measurement (S400), the humidity measurement may include measuring the humidity of the internal air of the fuel cell stack enclosure 20 and measuring the humidity of the external air of the fuel cell stack enclosure 20.

In the operation of performing measurement (S400), the temperature of the external air of the fuel cell stack enclosure 20 may be assumed to be a first temperature, and the temperature of the internal air of the fuel cell stack enclosure 20 may be assumed to be a second temperature. The humidity of the external air of the fuel cell stack enclosure 20 may be assumed to be a first humidity, and the humidity of the internal air of the fuel cell stack enclosure 20 may be assumed to be a second humidity.

Referring to FIGS. 2A, 3A, and 4, the method of operating the fuel cell stack sensor 210 may further include comparing the first temperature and the second temperature (S410, hereinafter ‘temperature comparison operation’). In the temperature comparison operation, it may be determined that the first temperature and the second temperature are the same or different. In the present disclosure, the fact that the temperatures are the same in the temperature comparison operation does not necessarily mean only the case in which the temperatures exactly match each other numerically, but may include not only the case in which the temperatures exactly match each other numerically but also the case in which a difference between the temperatures is within a predetermined error range.

Referring to FIG. 4, the method of operating the fuel cell stack sensor 210 may further include comparing the first humidity and the second humidity (S421 and S422). The comparing of the first humidity and the second humidity may be performed after the temperature comparison operation (S410). Depending on a result of the temperature comparison operation (S410), the method of operating the fuel cell stack sensor 210 may be changed.

When the first temperature and the second temperature are the same in the temperature comparison operation (S410), the method of operating the fuel cell stack sensor 210 may further include comparing the first humidity and the second humidity (S421, hereinafter ‘first humidity comparison operation’).

Referring to FIGS. 1 and 4, when the first temperature and the second temperature are different from each other in the temperature comparison operation (S410), the method of operating the fuel cell stack sensor 210 may further include determining whether the hydrogen vehicle 100 (FIG. 1) is restarted (S412). When the first temperature and the second temperature are different, the method of operating the fuel cell stack sensor 210 may further include comparing the first humidity and the second humidity (S422, hereinafter referred to as ‘second humidity comparison operation’).

Referring back to FIG. 4, the method of operating the fuel cell stack sensor 210 may further include an additional operation according to the result of the temperature comparison operation (S410) and the first humidity comparison operation (S421) or an additional operation according to the result of the second humidity comparison operation (S422).

Referring to FIGS. 1 and 4, the method of operating the fuel cell stack sensor 210 may further include comparing the first humidity and the second humidity again after a predetermined time has elapsed (hereinafter, a third humidity comparison operation, S423) when the first temperature and the second temperature are the same in the temperature comparison operation (S410) and the first humidity and the second humidity are the same in the first humidity comparison operation (S421). The predetermined time may be set as an arbitrary constant value on a system within the measurement controller 324.

Referring to FIGS. 1 and 2A, a route through which air A5 is introduced into the fuel cell stack 200 by the air supply system 110 and a route through which external air is introduced into the fuel cell stack enclosure 20 may be different, and because the air vent in the inlet 221 of the flow route of the fuel cell stack enclosure 20 does not pass through the humidifier 114, the humidity outside the fuel cell stack enclosure 20 and the humidity inside the fuel cell stack enclosure 20 may be the same. When the airtight inside the fuel cell stack 200 is broken, (i.e., when a leak has occurred) the humidified air A5 supplied into the fuel cell stack 200 may be cooled by the air cooler 113 and humidified by the humidifier 114, which may correspond to a case where the cooled and humidified air leaks into the fuel cell stack enclosure 20. Accordingly, the occurrence of a leak may be one of factors that cause the first humidity and the second humidity to be different and the first temperature and the second temperature to be different. Unless there is a special circumstance such as that an airtight inside the fuel cell stack 200 is broken, the first humidity and the second humidity may be the same, and the first temperature and the second temperature may be the same. Even though the first humidity, which is the humidity of the external air of the fuel cell stack enclosure 20, and the second humidity, which is the humidity of the internal air of the fuel cell stack enclosure 20, are the same, when the first humidity and the second humidity become different after a predetermined time has elapsed, the reason for the change in the second humidity may be that a leak occurs inside the fuel cell stack 200 and/or the fuel cell stack enclosure 20. When the hydrogen vehicle 100 is driving, the internal temperature of the fuel cell stack 200 may rise, and when a leak occurs in the fuel cell stack 200, air having a raised temperature is leaked inside the fuel cell stack enclosure 20. Therefore, leak occurrence may be one of the factors that cause the first temperature and the second temperature to be different.

Referring to FIGS. 2 and 4, when the first temperature and the second temperature are the same in the temperature comparison operation (S410), the first humidity and the second humidity are the same in the first humidity comparison operation (S421), and the first humidity and the second humidity are the same in the third humidity comparison operation (S423), the method of operating the fuel cell stack sensor 210 may further include providing the user with a signal indicating that both the fuel cell stack 200 and the fuel cell stack sensor 210 operate normally (S431, hereinafter referred to as ‘first normal operation determination operation’).

Referring to FIGS. 1, 2A, and 4, when the first temperature and the second temperature are the same in the temperature comparison operation (S410), the first humidity and the second humidity are the same in the first humidity comparison operation (S421), and the first humidity and the second humidity are different in the third humidity comparison operation (S423), the method of operating the fuel cell stack sensor 210 may further include determining that a leak has occurred and providing the user with a signal indicating that a leak has occurred inside the fuel cell stack 200 and/or the fuel cell stack enclosure 20 (S432, hereinafter ‘first internal leak determination operation’).

Referring to FIG. 4, when the first temperature and the second temperature are the same in the temperature comparison operation (S410) and the first humidity and the second humidity are different in the first humidity comparison step (S421), the method of operating the fuel cell stack sensor 210 may further include determining that the inside of the fuel cell stack sensor 210 is poisoned by VOCs and providing the user with a signal indicating that the fuel cell stack sensor 210 is poisoned by VOCs (S433, hereinafter referred to as a ‘VOC poisoning determination operation’).

Referring to FIGS. 1 and 4, when the first temperature and the second temperature are different in the temperature comparison operation (S410), the hydrogen vehicle 100 including the fuel cell stack sensor 210 is restarted (S412), and the first humidity and the second humidity are the same in the second humidity comparison operation (S422), the method of operating the fuel cell stack sensor 210 may further include providing the user with a signal indicating that the fuel cell stack 200 and/or the fuel cell stack sensor 210 is normally operating (S434, hereinafter, ‘second normal operation determination operation’).

In the case where the first temperature and the second temperature are different in the temperature comparison operation (S410), the hydrogen vehicle 100 of FIG. 1 including the fuel cell stack sensor 210 is restarted (S412), and the first humidity and the second humidity are different in the second humidity comparison operation (S422), the method of operating the fuel cell stack sensor 210 may further include determining that a leak has occurred inside the fuel cell stack 200 and/or the fuel cell stack enclosure 20 and providing the user with a signal indicating that a leak has occurred inside the fuel cell stack 200 and/or the fuel cell stack enclosure 20 (S435, hereinafter referred to as ‘second internal leak determination operation’).

When the method of operating the fuel cell stack sensor 210 includes the first normal operation determination operation S431 or the second normal operating determination operation S434, the method of operating the fuel cell stack sensor 210 may further include calculating a second hydrogen concentration value using the second temperature, the second humidity, and the first hydrogen concentration value (S441).

When the method of operating the fuel cell stack sensor 210 includes the VOC poisoning determination operation (S433), the method of operating the fuel cell stack sensor 210 may further include calculating a second hydrogen concentration value using the second temperature, the first humidity, and the first hydrogen concentration value (S442).

When the method of operating the fuel cell stack sensor 210 includes the first internal leak determination operation S432 or the second internal leak determination operation S435, the method of operating the fuel cell stack sensor 210 may further include generating a signal capable of controlling the operation of the hydrogen vehicle 100 by generating a diagnostic trouble code (DTC) (S443).

The above description is merely illustrative of the technical idea of the present disclosure, and various modifications and variations may be made without departing from the essential characteristics of the present disclosure by those with ordinary skill in the art to which the present disclosure pertains.

Therefore, the embodiments of the present disclosure are provided to explain the spirit and scope of the present disclosure, but not to limit them, so that the spirit and scope of the present disclosure is not limited by embodiments. The scope of protection of the present disclosure should be interpreted by the following claims, and all technical ideas within the scope equivalent thereto should be construed as being included in the scope of the present disclosure.

When using the fuel cell stack sensor according to the method disclosed in the present disclosure, it is possible to detect a hydrogen leak around the fuel cell stack, and furthermore, notify a user of whether VOC poisoning occurs in the sensor and whether there is a leak inside the fuel cell stack in real-time, thus preventing accidents due to problems occurring inside the vehicle in advance and promoting the safety of the vehicle user.

Hereinabove, although the present disclosure has been described with reference to embodiments and the accompanying drawings, the present disclosure is not limited thereto but may be variously modified and altered by those with ordinary skill in the art to which the present disclosure pertains without departing from the spirit and scope of the present disclosure claimed in the following claims.

What is claimed is:

1. A fuel cell stack sensor comprising:
a first substrate configured to measure at least one of an external temperature or an external humidity of a fuel cell stack enclosure including a fuel cell stack;
a second substrate configured to measure at least one of an internal temperature or an internal humidity of the fuel cell stack enclosure; and
a hydrogen sensing element disposed on the second substrate configured to detect hydrogen inside the fuel cell stack enclosure.

2. The fuel cell stack sensor of claim 1, further comprising:
a first temperature element disposed on the first substrate and configured to measure the external temperature of the fuel cell stack enclosure, and
a second temperature element disposed on the second substrate and configured to measure the internal temperature of the fuel cell stack enclosure.

3. The fuel cell stack sensor of claim 2, further comprising:
a first humidity element disposed on the first substrate and configured to measure the external humidity of the fuel cell stack enclosure; and
a second humidity element disposed on the second substrate and configured to measure the internal humidity of the fuel cell stack enclosure.

4. The fuel cell stack sensor of claim 3, further comprising a measurement controller configured to receive:
a first temperature value measured by the first temperature element;
a second temperature value measured by the second temperature element;
data including a first humidity value measured by the first humidity element; and
data including a second humidity value measured by the second humidity element.

5. The fuel cell stack sensor of claim 4, wherein the measurement controller is configured to further receive data including a hydrogen concentration value detected by the hydrogen sensing element.

6. The fuel cell stack sensor of claim 3, further comprising a measurement controller configured to control:
the first temperature element to measure the external temperature of the fuel cell stack enclosure;
the first humidity element to measure the external humidity of the fuel cell stack enclosure;
the second temperature element to measure the internal temperature of the fuel cell stack enclosure;
the second humidity element to measure the internal humidity of the fuel cell stack enclosure; and
the hydrogen sensing element to measure a hydrogen concentration inside the fuel cell stack enclosure.

7. The fuel cell stack sensor of claim 6, wherein the measurement controller is configured to:
calculate a first relative humidity value based on the external temperature and the external humidity; and
calculate a second relative humidity value based on the internal temperature and the internal humidity.

8. The fuel cell stack sensor of claim 6, wherein the measurement controller is configured to determine that volatile organic compound (VOC) poisoning has occurred in the fuel cell stack sensor when the external temperature and the internal temperature are the same and the external humidity and the internal humidity are different.

9. The fuel cell stack sensor of claim 6, wherein the measurement controller is configured to compare the external humidity and the internal humidity again after a predetermined time has elapsed when the external temperature and the internal temperature are the same and the external humidity and the internal humidity are the same, and
wherein the measurement controller is configured to determine that a leak has occurred inside the fuel cell stack enclosure when the external humidity and the internal humidity are different after the predetermined time has elapsed.

10. The fuel cell stack sensor of claim 6, wherein the measurement controller is configured to determine that a leak has occurred inside the fuel cell stack enclosure when the external temperature and the internal temperature are different and the external humidity and the internal humidity are different.

11. A method of operating a fuel cell stack sensor, the method comprising:
comparing a first temperature value indicating an external temperature of a fuel cell stack enclosure including a fuel cell stack with a second temperature value indicating an internal temperature of the fuel cell stack enclosure;
comparing a first humidity value indicating an external humidity of the fuel cell stack enclosure with a second humidity value indicating an internal humidity of the fuel cell stack enclosure; and
determining an operation of the fuel cell stack sensor based on comparison results of the first and second temperature values and of the first and second humidity values.

12. The method of claim 11, further comprising:
in response to determination that the first temperature value and the second temperature value are the same and the first humidity value and the second humidity value are the same, repeating, after a predetermined time period, comparing the first humidity value with the second humidity value . . .

13. The method of claim 11, wherein determining the operation of the fuel cell stack sensor comprises:
determining that the fuel cell stack sensor is poisoned by volatile organic compounds (VOCs) when the first temperature value and the second temperature value are the same, and the first humidity value and the second humidity value are different.

14. The method of claim 11, wherein determining the operation of the fuel cell stack sensor comprises:
in response to determining that the first temperature value and the second temperature value are different and the first humidity value and the second humidity value are the same, providing a user with a signal indicating that the fuel cell stack enclosure and the fuel cell stack sensor normally operating.

15. The method of claim 11, wherein determining the operation of the fuel cell stack sensor comprises:
in response to determining that the first temperature value and the second temperature value are different and the first humidity value and the second humidity value are different, determining that a leak has occurred inside the fuel cell stack enclosure.

16. The method of claim 12, wherein determining the operation of the fuel cell stack sensor comprises:
in response to determining that the first temperature value and the second temperature value are the same, and the first humidity value and the second humidity value are the same and, even after the predetermined time has elapsed, the first humidity value and the second humidity value are the same, providing a user with a signal indicating that an inside of the fuel cell stack enclosure and the fuel cell stack sensor are normally operating.

17. The method of claim 12, wherein determining the operation of the fuel cell stack sensor comprises:
in response to determining that the first temperature value and the second temperature value are the same, and the first humidity value and the second humidity value are the same and, after the predetermined time has elapsed, the first humidity value and the second humidity value are different, determining that a leak has occurred inside the fuel cell stack enclosure.

18. The method of claim 11, further comprising outputting, by the fuel cell stack sensor, a second hydrogen concentration value using the second temperature value, the second humidity value, and a first hydrogen concentration value corresponding to a hydrogen concentration inside the fuel cell stack enclosure when the operation of the fuel cell stack sensor is provided to a user with a signal indicating that an inside of the fuel cell stack enclosure and the fuel cell stack sensor are normally operating.

19. The method of claim 11, further comprising:
generating, by the fuel cell stack sensor, a diagnostic trouble code (DTC) to control a vehicle when the operation of the fuel cell stack sensor indicates that a leak has occurred inside the fuel cell stack enclosure.

20. The method of claim 11, further comprising:
outputting, by the fuel cell stack sensor, a second hydrogen concentration by using the second temperature value, the first humidity value, and a first hydrogen concentration value corresponding to a hydrogen concentration inside the fuel cell stack enclosure, when the operation of the fuel cell stack sensor indicates that the fuel cell stack sensor is poisoned by volatile organic compounds (VOCs).
